(19) United States
(12) Patent Application Publication
(10) Pub. No.: US 2003/0130563 A1
(43) Pub. Date: Jul. 10, 2003

(54) Hysteroscope port and methods

(76) Inventor: Randall A. Loy, Longwood, FL (US)

Correspondence Address: Allen, Dyer, Doppelt, Milbrath & Gilchrist, P.A., Suite 1401, 255 South Orange Avenue, P.O. Box 3791, Orlando, FL, 32802-3791, US

(21) Appl. No.: 10/304,531

(22) Filed: Nov. 26, 2002

Related U.S. Application Data
(63) Continuation-in-part of application No. 09/260,901, filed on Mar. 2, 1999, now Pat. No. 6,485,410.
(60) Provisional application No. 60/107,050, filed on Nov. 4, 1998.

(51) Int. Cl. A61B 1/00

(52) U.S. Cl. 600/114; 600/135

(57) ABSTRACT

A hysteroscope port includes a cannula member that has an elongated, generally cylindrical distal portion. The distal portion has a lumen that extends between a proximal opening and a distal opening. The distal portion is dimensioned for insertion through a human vagina and into a cervix uteri. The distal portion has a surface that is adapted to resist slippage against vaginal and cervical tissue, and has a cervix-engaging section proximal of the distal opening having a threaded outer surface for increasing resistance to slippage against the cervical canal.

### CROSS-REFERENCE TO RELATED APPLICATION

This application is a continuation-in-part of co-pending application Ser. No. 09/260,901, filed Mar. 2, 1999, which in turn claims priority to provisional application Serial No. 60/107,050, filed Nov. 4, 1998. which are commonly owned with the present invention and which are incorporated herein by reference.

### BACKGROUND OF THE INVENTION

1. Field of the Invention

The present invention relates to surgical devices and methods, and, more particularly, to devices and methods for providing an instrument portal into the uterus.

2. Description of Related Art

The evolution of surgical procedures has been along a path of decreasing invasiveness. Such changes have been advanced by the introduction of systems permitting external visualization of procedures and devices that are externally manipulable.

In the obstetrical/gynecological arena, for example, access into the uterus and fallopian tubes can be achieved by inserting a speculum into the vagina and performing a dilatation of the cervix if necessary or desired, such as with the use of cervical clamps. Visualization and manipulation instruments can then be inserted through the speculum opening to carry out a desired procedure. Among the devices currently used is a flexible hysteroscope, which may include visualization means such as fiber optic illumination and viewing elements and forceps-type grasping jaws.

A particular problem with the current devices and methods, however, is that multiple placements of the speculum are usually required, and the cervix is difficult to restrain in a dilated position, as it is biased to a closed position.

Laparoscopic trocars are known in the art for insertion through tissue to obtain access to an internal organ. Their design is predicated on the cylindrical portion of the trocar remaining in place in the skin, and it is typically desired to keep the hole in the skin as small as possible. Such trocars have been designed with balloons, but these balloons are for retaining that section of the trocar within the body's interior.

### SUMMARY OF THE INVENTION

It is therefore an object of the present invention to provide a device for passing an instrument into the uterus through the cervix.

It is an additional object to provide such a device that retains the cervix in a dilated state.

It is a further object to provide such a device whose insertion is not damaging to the bodily tissue.

It is another object to provide a method of performing a gynecological procedure.

An additional object is to provide a method of making a device for passing instrument into a uterus.

These objects and others are achieved by the device and methods of the present invention. The device comprises an elongated generally cylindrical cannula having a proximal and a distal opening and a lumen therebetween. The cannula is dimensioned for insertion through a vagina to penetrate a cervix. Means are positioned adjacent the distal end of the cannula for dilating the cervix and for maintaining the cervix in a dilated state. The dilatation means are manipulable between a narrowed insertion and withdrawal position and an expanded dilatation position. The dilatation means have a surface adapted to resist slippage against the cervical surface.

The device also comprises an introducer having a generally cylindrical distal portion dimensioned for insertion through the cannula lumen and sufficiently long to extend beyond the cannula's distal end. The introducer's distal tip is tapered for ease of insertion, but is preferably smooth to prevent tissue damage. The introducer also has a proximal portion adapted to protrude from the cannula's proximal opening and having means for being grasped for insertion into and removal from the cannula.

In an alternate embodiment of the invention, a hysteroscope port comprises a cannula member that has an elongated, generally cylindrical distal portion. The distal portion has a lumen that extends between a proximal opening and a distal opening. The distal portion is dimensioned for insertion through a human vagina and into a cervix uteri. The distal portion has a surface that is adapted to resist slippage against vaginal and cervical tissue, and has a cervix-engaging section proximal of the distal opening having a threaded outer surface.

The features that characterize the invention, both as to organization and method of operation, together with further objects and advantages thereof, will be better understood from the following description used in conjunction with the accompanying drawing. It is to be expressly understood that the drawing is for the purpose of illustration and description and is not intended as a definition of the limits of the invention. These and other objects attained, and advantages offered, by the present invention will become more fully apparent as the description that now follows is read in conjunction with the accompanying drawing.

### BRIEF DESCRIPTION OF THE DRAWINGS

FIG. 1 is a side perspective view the device of the present invention in the insertion configuration.

FIG. 2 is a side perspective view of the device in the dilatation configuration.

FIG. 3 is a side perspective view of an alternate embodiment of the device of the present invention.

### DETAILED DESCRIPTION OF THE PREFERRED EMBODIMENTS

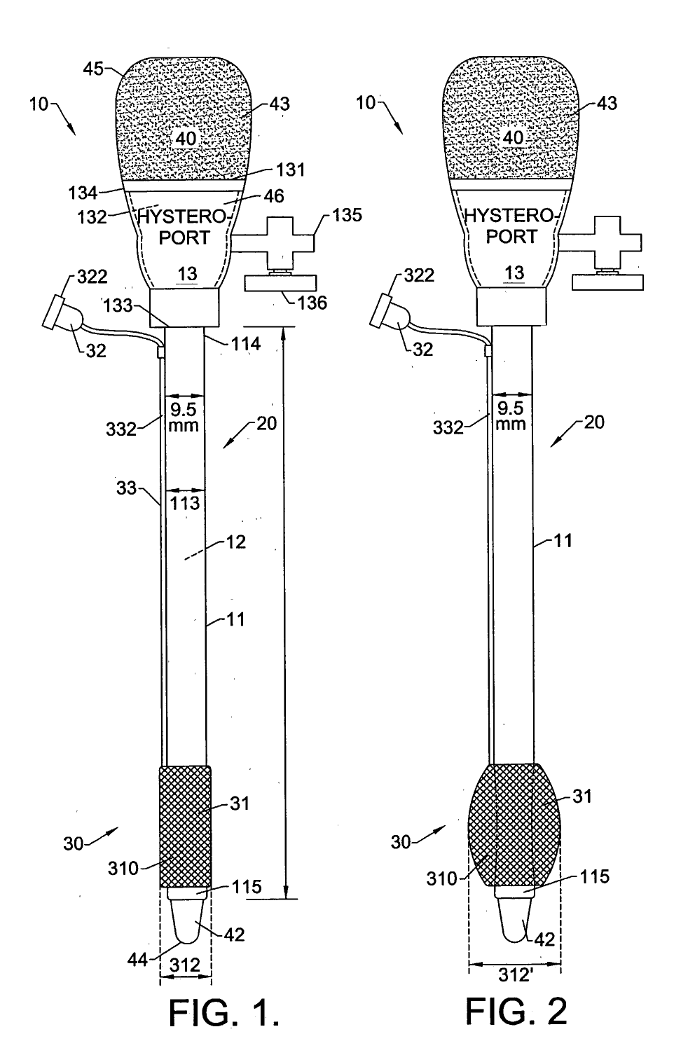

A description of the preferred embodiments of the present invention will now be presented with reference to FIGS. 1-3.

The hysteroscope port 10 comprises three elements: a cannula 20, an inflator mechanism 30, and an introducer 40.

The cannula 20 comprises a generally cylindrical distal portion 11 having a length 112 sufficient to penetrate a vagina and extend beyond the cervix and a diameter 113 dimensioned for comfortable insertion, e.g., 9.5 mm, although this is not intended as a limitation. The lumen 12 is dimensioned to permit the passage of a desired instrument therethrough.

Adjacent the distal portion's 11 proximal end 114 is a portal element 13 that has a proximal opening 131 communicating with an entry space 132 that communicates at its distal end 133 with the lumen 12. The portal element 13 tapers outward from its distal end 133 to its proximal end 134. Within the entry space 132 is affixed means for isolating the lumen from the exterior environment, which has means for admitting a desired instrument. These means may comprise, for example, means as are known in the art such as a flapper valve or rubber seal (not shown), although these are not intended as limitations.

Extending radially from the side of the portal element 13 is a valved influx port 135 through which a fluid such as distension medium may be added into the entry space 132. Handle 136 operates a valve for permitting or preventing access through the port 135.

The inflator mechanism 30 comprises a generally annular inflatable balloon member 31 affixed in surrounding relation to the cannula's distal portion 11 adjacent the distal end 115. The balloon 31 preferably has a coarse outer surface 310 adapted to frictionally engage the cervix and to this end may be, for example, striated and/or ribbed.

Inflation of the balloon 31 is achieved by injecting a fluid through an inflation port 32 having an opening 322 communicating with the lumen 332 of a line 33 that is positioned adjacent at least a portion of the length of the cannula's distal portion 11. The line's lumen 332 in turn communicates with the interior of the balloon 31 so that inflation may be achieved from an insertion configuration (FIG. 1), wherein the balloon 31 has substantially the same diameter 312 as that 113 of the cannula's distal portion 11, to a dilatation configuration (FIG. 2), wherein the balloon 31 is inflated so that its outer surface bulges to a larger diameter 312′ than that 113 of the cannula's distal portion 11.

The device 10 further comprises an introducer 40 for facilitating the insertion of the cannula 20 into position. The introducer 40 includes a generally cylindrical distal portion (not shown; interior to cannula distal portion 11) dimensioned for insertion through the cannula's lumen 12. The distal tip 42 tapers to a blunt end 44 to ease insertion without damaging tissue. Preferably at least the distal tip 42 comprises a material sufficiently resilient, such as a rubber or plastic, also to avoid damaging tissue.

The introducer's proximal portion 43 is widened to prevent its passage into the cannula's lumen 12 and has a distal section 46 dimensioned to fit within the portal's entry space 132. The proximal section 45 of the proximal portion 43 is dimensioned to protrude proximal of the portal 13 so that it may be gripped for removal from the cannula 20.

The method of using the device 10 comprises the steps of inserting the device 10, with the introducer 40 in place inside the cannula 20, into a vagina sufficiently far that the introducer's distal tip 42 protrudes into the uterus and the balloon 31, in the insertion configuration, spans the cervix.

By forcing a fluid under pressure through the inflation port's opening 322 and through the line 33 into the balloon 31 until it is sufficiently inflated to dilate the cervix (FIG. 2), the coarse surface 310 frictionally engaging the cervix to retain it thereagainst. Next the introducer 40 is removed from the cannula 20.

Once the desired surgical procedure has been completed, the balloon 31 is deflated by releasing the pressure from the inflation port's opening 322, and the cannula 20 is removed.

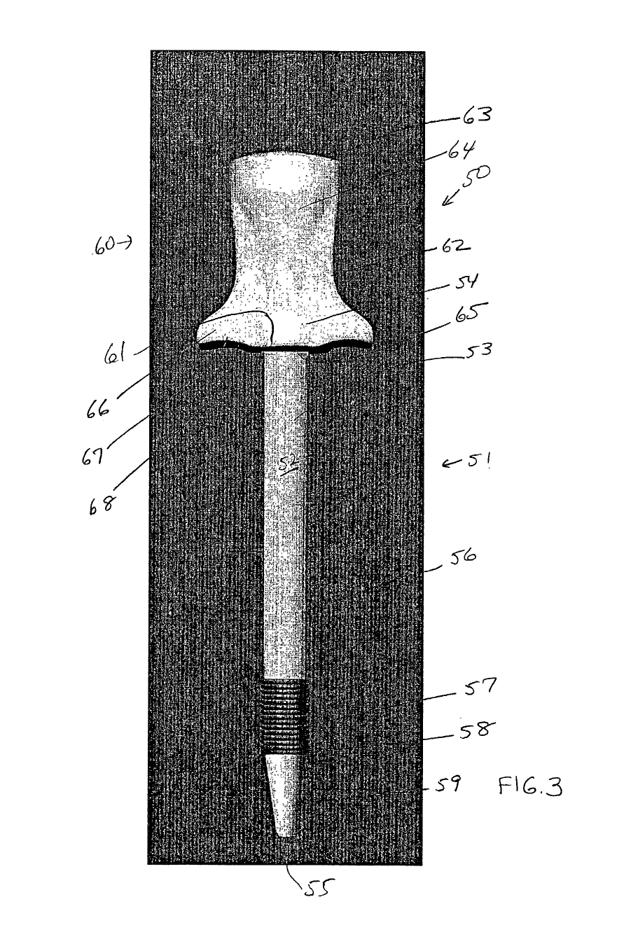

In an alternate embodiment (FIG. 3), the hysteroscope port 50 comprises a cannula member 51 that has an elongated, generally cylindrical distal portion 52 having a lumen 53 extending between a proximal opening 54 and a distal opening 55. The lumen 53 is dimensioned sufficiently large to permit a passage of a desired instrument therethrough. The distal portion 52 is itself dimensioned for insertion through a human vagina and into a cervix uteri. The distal portion 52 has a surface adapted to resist slippage against vaginal and cervical tissue, for example, a mesh 56 such as is known in the art for use as a stent surface.

The distal portion 52 of the cannula member 51 further has a cervix-engaging section 57 that is positioned proximal of the distal opening 55. This cervix-engaging surface 57 has, for example, a threaded outer surface 58, positioned approximately 2.5 cm from the distal end and approximately 2.5 cm long. The cervix-engaging surface 57 resists movement against the tissue and serves to maintain the cannula member 51 within the canal.

The distal portion 52 also has an introduction section 59 that is adjacent and distal of the cervix-engaging section 57. The introduction section 59 tapers downward distally toward the distal opening 55 to a tip, for facilitating introduction into the vagina and the cervix uteri.

The cannula member 51 further comprises a portal element 60 joining the distal portion 52 at a proximal end 61 thereof. The portal element 59 has a wall 62 and a proximal opening 63 leading to an entry space 64 in communication with the lumen 53 at the distal end 65. Preferably the entry space 64 tapers outward in a proximal direction.

The portal element 59 further has a grip section 66 at the distal end 65 that flares outward in a distal direction and has finger depressions 67 on a distal face 68 for facilitating gripping.

In use, the hysteroscope port 50 is inserted through the vaginal canal into the cervix uteri, the grip section 66 is grasped, and a hysteroscope is passed through the cannula member's lumen 53 and out the distal opening 55 in order to visualize the uterus. Distension fluid may also be introduced through the lumen 53 in order to improve visualization.

Once the hysteroscope is introduced, another surgical instrument may also be inserted in order to perform a desired procedure, after which both the instrument and the hysteroscope are withdrawn.

It may be appreciated by one skilled in the art that additional embodiments may be contemplated, including various other devices for achieving dilatation and for achieving frictional engagement with the cervical wall.

In the foregoing description, certain terms have been used for brevity, clarity, and understanding, but no unnecessary limitations are to be implied therefrom beyond the requirements of the prior art, because such words are used for description purposes herein and are intended to be broadly construed. Moreover, the embodiments of the apparatus illustrated and described herein are by way of example, and the scope of the invention is not limited to the exact details of construction.

What is claimed is:

1. A hysteroscope port comprising a cannula member having an elongated, generally cylindrical distal portion having a lumen extending between a proximal opening and a distal opening, the distal portion dimensioned for insertion through a human vagina and into a cervix uteri, the distal portion having a surface adapted to resist slippage against vaginal and cervical tissue, the distal portion having a cervix-engaging section proximal of the distal opening having a threaded outer surface.

2. The hysteroscope port recited in claim 1, wherein the lumen is dimensioned sufficiently large to permit a passage of a desired instrument therethrough.

3. The hysteroscope port recited in claim 1, wherein the cannula member further comprises a portal element joining the distal portion at a proximal end thereof, the portal element having a wall and a proximal opening leading to an entry space in communication with the distal portion lumen at a distal end, the entry space tapering outward in a proximal direction.

4. The hysteroscope port recited in claim 3, wherein the portal element further has a grip section at a distal end thereof, the grip section flaring outward in a distal direction and having finger depressions on a distal face for facilitating gripping.

5. The hysteroscope port recited in claim 1, wherein the distal portion further has an introduction section adjacent and distal of the cervix-engaging section, the introduction section tapering downward distally toward the distal opening for facilitating introduction into the vagina and the cervix uteri.

6. The hysteroscope port recited in claim 1, wherein a portion of the distal portion surface comprises a mesh.

7. A method for visualizing a human uterus comprising the steps of:
inserting a generally cylindrical cannula member through a vaginal canal into a cervix uteri of a patient, the cannula member having an elongated, generally cylindrical distal portion having a lumen extending between a proximal opening and a distal opening, the distal portion dimensioned for insertion through a human vagina and into a cervix uteri, the distal portion having a surface adapted to resist slippage against vaginal and cervical tissue, the distal portion having a cervix-engaging section proximal of the distal opening having a threaded outer surface; and
passing a distal end of a hysteroscope through the cannula member lumen and out the cannula member distal opening, for visualizing a uterus of the patient.

8. The method recited in claim 7, wherein:
the inserting step comprises gripping a portal element of the cannula member, the portal element joining the distal portion at a proximal end thereof, the portal element having a wall and a proximal opening leading to an entry space in communication with the distal portion lumen at a distal end, the entry space tapering outward in a proximal direction; and
further comprising the step of gripping the portal element while performing the passing step.

9. The method recited in claim 8, wherein the portal element further has a grip section at a distal end thereof, the grip section flaring outward in a distal direction and having finger depressions on a distal face, and the gripping step comprises gripping the grip section.

10. The method recited in claim 7, wherein the cannula member distal portion further has an introduction section adjacent and distal of the cervix-engaging section, the introduction section tapering downward distally toward the distal opening to form a tip, and wherein the inserting step comprises introducing the introduction section into the vagina and the cervix uteri.

11. The method recited in claim 7, further comprising the step of introducing a distension fluid through the cannula member lumen into the uterus following the inserting step.

12. The method recited in claim 7, further comprising the steps, following the passing step, of:
inserting a surgical instrument through the cannula member lumen;
performing a desired procedure with the instrument;
withdrawing the instrument from the cannula member lumen; and
withdrawing the cannula member from the cervix uteri and the vagina.
